(19) United States
(12) Patent Application Publication
(10) Pub. No.: US 2021/0146081 A1
(43) Pub. Date: May 20, 2021

(54) EMERGENCY RESUSCITATION SYSTEM WITH REMOTE CONTROL

(71) Applicant: Weinmann Emergency Medical Technology GMBH + Co., KG, Hamburg (DE)

(72) Inventors: Patrick NOLDT, Tornesch (DE); Matthias PULLA, Hamburg (DE); Carsten LÜTJENS, Hamburg (DE)

(21) Appl. No.: 17/135,045

(22) Filed: Dec. 28, 2020

Related U.S. Application Data
(63) Continuation-in-part of application No. 15/692,732, filed on Aug. 31, 2017, now abandoned.

(30) Foreign Application Priority Data
Feb. 22, 2017 (DE) 20 2017 000 938.2

(51) Int. Cl. A61M 16/00; A61M 16/08; A61M 16/06

(52) U.S. Cl. CPC A61M 16/024; A61M 16/0875; A61M 2209/01; A61M 16/0084; A61M 16/06

(57) ABSTRACT

A resuscitation system is provided with a resuscitation device that is arranged away from the patient. Moreover, the resuscitation device is connected to a nozzle of a patient interface in an air-conducting manner by a respiratory gas tube. Near the patient, a remote control for the resuscitation device is arranged in the region of the patient interface. The remote control includes a casing with an upper side and a lower side. The upper side and/or the lower side are produced, at least in sections, from hard plastic. The remote control further has at least one operating element and comprises an electronic printed circuit board or, at least, an electronic component.

### CROSS-REFERENCE TO RELATED APPLICATIONS

The present application is a continuation-in-part of co-pending U.S. patent application Ser. No. 15/692,732, filed Aug. 31, 2017, which claims priority of DE 20 2017 000 938.2, filed Feb. 22, 2017, the priority of these applications is hereby claimed and these applications are incorporated herein by reference.

### BACKGROUND OF THE INVENTION

The invention relates to an emergency resuscitation device comprising a remote control, by means of which respiratory gas volumes for the patient are triggered.

A multiplicity of different resuscitation devices and resuscitation bags have already been proposed and are known to a person skilled in the art. Resuscitation bags merely consist of a mask with a hand-operated bag.

Resuscitation devices consist of the resuscitation device with a pressurized gas source, a respiratory gas tube and a patient interface, such as a mask, that is fastened to the tube.

A disadvantage of the bag resuscitation is that the user's whole attention is directed to the bag resuscitation and said user is usually only able to carry out said bag resuscitation for a short period of time.

A disadvantage of resuscitation devices is that the adjustment options for the resuscitation are only available on the resuscitation device. However, the user must turn their attention to the patient and press the patient interface onto the patient's face.

Certain emergency resuscitation devices (resuscitators), the type of which is described in e.g. U.S. Pat. No. 8,800,562 B2, have a relatively compact structure and can therefore be placed directly onto the patient interface. Such emergency resuscitation devices are illustrated in FIG. 1 of the present application as prior art. The emergency resuscitation device has all pneumatic, mechanical and/or electronic components that are required for resuscitation. Moreover, it has a manual operating element which the user must actuate in order to trigger a respiratory gas volume.

A disadvantage of these emergency resuscitation devices is that a relatively large and heavy device is connected directly to the mask. As a result, the facial field of the patient is partly covered, even though the aider/user must precisely monitor the face of the patient. It is also disadvantageous that the emergency resuscitation device still requires a relatively large amount of space above the mask. In some accident situations, for example in the case of buried patients, it is precisely this space that is not available. It is also disadvantageous that the emergency resuscitation device is relatively large and heavy. Size and weight in conjunction with the high structure cause the rollover tendency to be high. Therefore, a second aider is required, who secures and holds the emergency resuscitation device. It is likewise disadvantageous that the manual operating element is oriented in the region of the back of the hand of the aider and hence precisely in the opposite direction to the patient.

This is because the aider/user must affix the patient interface on the face of the patient with both hands (a so-called C-grip) in order to effectively preclude leakages at this position. Therefore, he is unable to actuate a manual operating element that is situated in the region of the back of the hand without neglecting the affixment of the mask.

In the case of intubated patients, there always is a risk of injury by displacing the tube. The aider/user must continuously monitor the correct seat of the tube and, moreover, take care that the tube is not displaced.

Emergency resuscitation devices, the type of which is described in U.S. Pat. No. 8,800,562 B2, are therefore unsuitable for use with a tube. The displacement of the tube is even probable on account of the dimensions and the arrangement of these emergency resuscitation devices. In the case of emergency use, a contamination of the resuscitation devices with dirt, germs, dust and moisture can seldom be avoided. Therefore, resuscitation devices and the operating elements of the devices, moreover, should be able to be cleaned without problems using conventional disinfection means.

### SUMMARY OF THE INVENTION

It is therefore an object of the present invention to specify a remote control for an emergency resuscitation device, which can be arranged in the vicinity of the airways of the patient and which has a very compact structure and by means of which the aider easily triggers respiratory gas volumes for the patient by hand, without neglecting the seal and the seat of the patient interface on the patient. It is also an object of the present invention to specify a remote control for an emergency resuscitation device, which has a robust structure and which is sealed against dirt, liquids and dust and which can easily and repeatedly be disinfected.

In view of the aforementioned objects, the invention comprises a resuscitation device comprising a tube and a patient interface and a remote control for the resuscitation device which can be arranged in the vicinity of the airways of the patient, namely on the patient interface, wherein the remote control has a very compact structure and lies in the region of the palm of the hand or of the fingers when the aider affixes the patient interface. By means of the remote control, the aider can easily trigger respiratory gas volumes for the patient by hand, without neglecting the seal of the patient interface on the patient.

In view of the aforementioned objects, the invention in particular comprises a resuscitation system comprising a resuscitation device which is arranged away from the patient and connected to the nozzle of a patient interface in an air-conducting manner by means of a respiratory gas tube, wherein, near the patient, a remote control for the resuscitation device is arranged in the region of the patient interface, wherein the remote control comprises a casing with an upper side and a lower side and the upper side and/or the lower side are produced, at least in sections, from hard plastic, wherein the remote control comprises at least one operating element and comprises an electronic printed circuit board or, at least, an electronic component, wherein the component is activated by actuating the operating element in order to produce a control signal for the resuscitation device, wherein the operating element has a surface which, optically and/or haptically, is distinct from the remainder of the remote control, wherein a direction of main extent of the remote control is aligned substantially parallel to the face of the patient, wherein the operating element extends through a wall in the casing upper side, for the purposes of which the casing has a recess, and the operating element has an interior region and an exterior region, wherein the interior region of the operating element is arranged within the casing and surrounded by the casing, characterized in that the remote control is detachably connected to the nozzle of the patient interface, wherein the exterior region of the operating element has a substantially areal configuration and the exterior region of the operating element is fitted into the casing upper side and sealed by a sealing unit in such a way that interlocking and/or cohesive affixment or coverage of the operating element relative to the casing upper side of the casing is achieved and a substantially dust-tight and/or watertight connection is obtained between the operating element and the casing, wherein the interior region of the operating element comprises a contact region which establishes a connection between the operating element and the component when the operating element is pressed down and a control signal is transmitted to the resuscitation device in order to trigger at least one respiratory gas volume.

In view of the aforementioned objects, the invention in particular also comprises a resuscitation system comprising a resuscitation device which is arranged away from the patient and connected to the nozzle of a patient interface in an air-conducting manner by means of a respiratory gas tube, wherein, near the patient, a remote control for the resuscitation device is arranged in the region of the patient interface, wherein the remote control comprises a casing with an upper side and a lower side and the upper side and/or the lower side are produced, at least in sections, from hard plastic and wherein the upper side and/or the lower side have a shaping, at least in sections, which is based on a contour of the patient interface or of the hand of a user, wherein the remote control comprises at least one operating element and comprises an electronic printed circuit board or, at least, an electronic component, wherein the component is activated by actuating the operating element in order to produce a control signal for the resuscitation device, wherein the operating element has a surface which, optically and/or haptically, is distinct from the remainder of the remote control, wherein the casing upper side has a contact region that is provided for coupling with the casing lower side, wherein the casing upper side and the casing lower side, in an assembled state, are securely connected to one another in a substantially watertight and/or dust-tight manner, wherein the casing of the remote control has a height HF and a length LF, wherein the length LF is greater than the height HF, as a result of which the remote control has a substantially areal configuration and a main direction of extent of the remote control is aligned substantially parallel to the face of the patient, wherein the operating element extends through a wall in the casing upper side, for the purposes of which the casing has a recess, and the operating element has an interior region and an exterior region, wherein the exterior region of the operating element is situated above, or level with, the upper side of the casing and the interior region of the operating element is arranged within the casing and surrounded by the casing, wherein the exterior region of the operating element has a substantially areal configuration and the exterior region of the operating element is fitted into the casing upper side and sealed by a sealing unit in such a way that interlocking and/or cohesive affixment or coverage of the operating element relative to the casing upper side of the casing is achieved and a substantially dust-tight and/or watertight connection is obtained between the operating element and the casing, wherein the interior region of the operating element comprises a contact region which establishes a connection between the operating element and the component when the operating element is pressed down and a control signal is transmitted to the resuscitation device in order to trigger at least one respiratory gas volume.

In a functional state, the resuscitation device that is arranged away from the patient may be arranged at a maximum distance of one tube length from the patient.

An operating element is preferably arranged in the main direction of extent.

An economical implementation of the invention is assisted by virtue of the remote control being connected to the patient interface in a detachable and reusable manner. As a result, the remote control can often be reused, while the patient interface may be a disposable article.

Substantially dust-tight and/or watertight within the meaning of the invention means that an ingress of dust and/or spray water is avoided in most cases if there is non-permanent exposure. However, extreme environmental conditions may still allow dust or water to enter.

At least in the region of the recess for the nozzle, the upper side and/or the lower side have a shaping which is based on a contour of the patient interface (e.g. the nozzle).

A simple operation is assisted by virtue of the exterior region of the operating element having a substantially areal configuration and having sections, the main direction of extent of which is arranged substantially in the direction of the casing upper side.

Good sealing is assisted by virtue of the sealing unit having an integral embodiment with the operating element and/or with the casing.

Simple manufacturing is assisted by virtue of the sealing unit forming a cohesive connection with the casing upper side and, proceeding from the casing upper side, completely covering the operating element.

Simple handling is assisted by virtue of the area of the casing upper side of the remote control being less than 60 cm².

Simple manufacturing and a good seal is assisted by virtue of the upper part of the casing consisting of a material with a relatively high Shore hardness, into which the operating element has been inserted, and a material with a relatively low Shore hardness being completely pasted or molded over the operating element.

Simple manufacturing and a simple operability is assisted by virtue of the sealing unit being produced from a soft plastic using the 2K process, said soft plastic being molded onto the hard-plastic casing of the remote control.

A simple operability is assisted by virtue of the operating element being configured as a touch sensitive (sensor) surface (touchscreen).

An individual operability is assisted by virtue of two operating elements being arranged in the casing of the remote control.

The two operating elements can be used in an alternative or complementary manner in order to trigger the control pulse for the resuscitation device for releasing a defined respiratory gas volume.

The two operating elements can alternatively trigger defined respiratory gas volumes of different dimensions.

An ergonomically expedient operability is assisted by virtue of the at least one operating element being configured and embodied in the remote control in such a way that an aider, if he affixes the patient interface over the airways of the patient with the typical C-grip, is able to actuate the operating element, in particular with the lower side of the fingers or the ball of the thumb.

An operability is also assisted by virtue of the length of the remote control undershooting the length of the mask.

A simple operability is also assisted by virtue of the height of the remote control HF being less than the height of the mask HM.

A safe application is also assisted by virtue of the assembly consisting of remote control and mask or PI only being as high as the patient interface on its own.

A simple structure is assisted by virtue of the remote control having a recess in the casing, said recess being dimensioned in such a way that the nozzle of the mask or of the PI can be introduced and the remote control is plugged onto the nozzle and therefore arranged near the mask.

A simple structure is also assisted by virtue of the remote control having a height which is dimensioned in such a way that the nozzle of the mask passes completely through the remote control and there still is enough free space on the nozzle in order to connect the tube.

A safe structure is assisted by virtue of the remote control comprising a plurality of holding elements in the recess, said holding elements serving to affix the remote control on the nozzle and latching or clamping the remote control onto the nozzle.

Simple manufacturing is assisted by virtue of the holding elements being molded onto the casing parts as a soft component in the 2k process.

Simple manufacturing is also assisted by virtue of the nozzle tapering conically such that the remote control is clamped onto the nozzle.

A safe application is also assisted by virtue of at least one indicating means being arranged in the region of the casing of the remote control.

A simple application is assisted by virtue of the remote control weighing less than 700 grams.

A simple application is also assisted by virtue of the remote control weighing less than 300 grams.

A simple application is also assisted by virtue of the remote control transmitting the control signals wirelessly to the resuscitation device.

A robust application is assisted by virtue of the remote control transmitting the control signals in a wired manner to the resuscitation device and the cable being guided out of the casing in the region of the upper side and the cable being sealed in relation to the casing in a substantially dust-tight and/or watertight manner and the cable having a kink protector adjacent to the casing.

The invention also relates to a remote control for use with a resuscitation device, wherein the resuscitation device is arranged away from the patient and connected to the nozzle of a patient interface in an air-conducting manner by means of a respiratory gas tube, wherein, near the patient, the remote control is arranged in the region of the patient interface, wherein the remote control comprises a casing with an upper side and a lower side and the upper side and/or the lower side are produced, at least in sections, from hard plastic and wherein the upper side and/or the lower side have a shaping, at least in sections, which is based on a contour of the patient interface or of the hand of a user, wherein the remote control comprises at least one operating element and comprises an electronic printed circuit board or, at least, an electronic component, wherein the component is activated by actuating the operating element in order to produce a control signal for the resuscitation device, wherein the operating element has a surface which, optically and/or haptically, is distinct from the remainder of the remote control, wherein the operating element extends through a wall in the casing upper side, for the purposes of which the casing has a recess, and the operating element has an interior region and an exterior region, wherein the exterior region of the operating element is situated above, or level with, the upper side of the casing and the interior region of the operating element is arranged within the casing and surrounded by the casing, characterized in that the exterior region of the operating element has a substantially areal configuration and the exterior region of the operating element is fitted into the casing upper side and sealed by a sealing unit in such a way that interlocking and/or cohesive affixment or coverage of the operating element relative to the casing upper side of the casing is achieved and a substantially dust-tight and/or watertight connection is obtained between the operating element and the casing, wherein the interior region of the operating element comprises a contact region which establishes a connection between the operating element and the component when the operating element is pressed down and a control signal is transmitted to the resuscitation device in order to trigger at least one respiratory gas volume.

The invention also relates to a remote control, configured and embodied for use in a resuscitation system according to the invention.

The invention also relates to a resuscitation device, configured and embodied for use in a resuscitation system according to the invention.

The invention also relates to a patient interface, configured and embodied for use in a resuscitation system according to the invention.

The resuscitation system for the first time provides the aider with the option of manually triggering a respiratory gas volume—by means of the remote control—without interrupting the two-handed affixment of the patient interface.

Compared to a resuscitator, less space is required in the region of the face since the apparatus is smaller than a commercially available resuscitator or a resuscitation bag. Consequently, resuscitation is also possible in rough terrain, and in the case of trapped or buried persons.

The lever effect on the patient interface and the risk of producing leakage between the patient interface and the face are significantly lower since the center of gravity is close to the patient.

The various features of novelty which characterize the invention are pointed out with particularity in the claims annexed to and forming a part of the disclosure. For a better understanding of the invention, its operating advantages, specific objects attained by its use, reference should be had to the drawings and descriptive matter in which there are illustrated and described preferred embodiments of the invention.

### BRIEF DESCRIPTION OF THE DRAWING

In the drawing:

FIG. 1 shows a prior art resuscitation device;

FIG. 2 shows a resuscitation system according to the present invention;

FIG. 3 shows another view of the inventive system;

FIG. 4 is a view from above of a mask and remote control;

FIG. 5 is a cross section of the remote control;

FIG. 6 shows additional details of the remote control;

FIG. 7 shows a device for carrying out ultrasonic welding;

FIG. 8 shows an enlarged illustration of a welding point for connecting a lower housing part to an upper housing part;

FIG. 9 illustrates in a cross-sectional view the connection of a lower housing part to an upper housing part by means of ultrasonic welding;

FIG. 10 shows an enlarged illustration of the arrangement according to FIG. 9 in the area of the weld;

FIG. 11 shows a representation of the combination of the lower housing part and the upper housing part;

FIG. 12 shows a plan view of a device with a lower housing part and an upper housing part;

FIG. 13 shows a perspective illustration of the arrangement according to FIG. 12; and

FIG. 14 shows a cross section to illustrate a support for the remote control on the collar of a breathing mask.

### DETAILED DESCRIPTION OF THE INVENTION

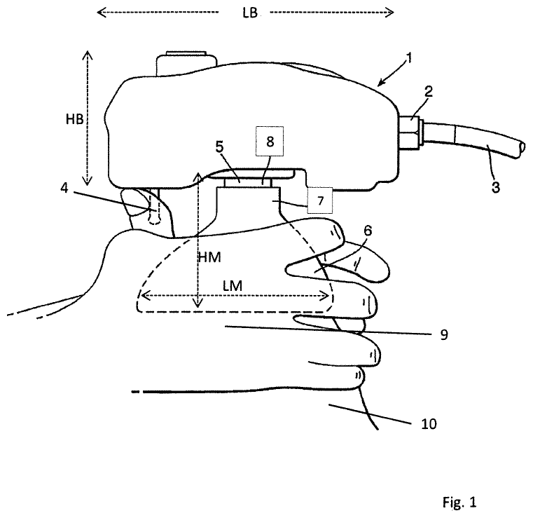

FIG. 1 shows an emergency resuscitation device from the prior art as per U.S. Pat. No. 8,800,562 B2. The resuscitation device 1 is connected to a respiratory gas tube 3 by way of a tube connector 2. By way of an adapter 5, it is connected to the nozzle 8 of the patient interface in an air-conducting manner. The patient interface 6 merges into the nozzle 8 by way of a connection part 7. The operating element 4 is situated on the lower side of the resuscitation device—and hence facing away from the palms of the aider. The user can actuate the operating element with a thumb of his hand 9 and trigger the resuscitation. Moreover, the typical grip, with which the aider affixes the patient interface 6 over the airways of the patient 10, is depicted. It is possible to recognize that the length of the resuscitation device LB significantly exceeds the length of the mask LM. Therefore, the resuscitation device partly covers the face of the patient. Moreover, it is possible to recognize that the height of the resuscitation device HB approximately corresponds to the height of the mask HM. The structure consisting of resuscitation device and mask is therefore twice as high as the patient interface (PI) or the mask itself.

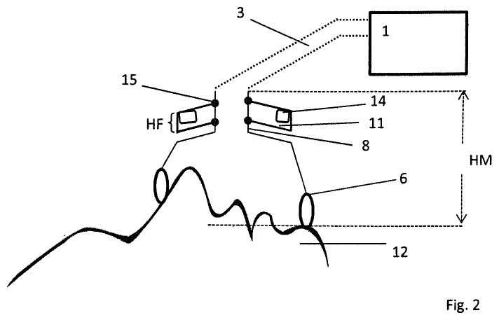

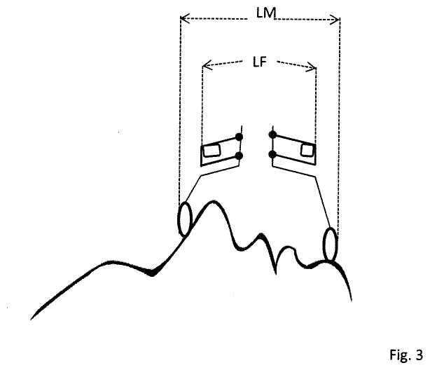

FIG. 2 and FIG. 3 show the system according to the invention. The resuscitation device 1 is arranged away from the patient and connected to the nozzle 8 of the mask or of the PI in an air-conducting manner by means of a respiratory gas tube 3. The patient interface 6 merges into the nozzle 8. At least one operating element 14, by means of which the user triggers the resuscitation with a finger of his hand 9 or the ball of his hand, is situated on the upper side of the remote control 11. The remote control is configured and embodied to transmit the signal to the resuscitation device. What is moreover depicted is that, if the aider affixes the patient interface 6 over the airways of the patient 10 by means of the typical C-grip, the index finger or middle finger and thumb, in particular, lie on the operating element 14. It is possible to recognize that the length of the remote control LF drops below the length of the mask LM. Therefore, the face 12 of the patient is not covered by the remote control 11. Moreover, it is possible to identify that the height of the remote control HF is significantly lower than the height of the mask HM. The remote control preferably has such dimensions that it is covered by both hands of the aider in the C-grip, in most cases completely covered by the hands, or surrounded by the hands. Here, the remote control is plugged onto the nozzle 8 and therefore arranged near the mask. The structure consisting of remote control and mask or PI is therefore always only as high as the patient interface on its own. Accordingly, the center of gravity of the structure is always close to the patient. The remote control 11 has a recess 16, which is dimensioned in such a way that the nozzle 8 of the mask can be inserted. For the purposes of affixing the remote control 11 to the nozzle 8, the remote control comprises a plurality of holding elements 15 in the recess 16, said holding elements latching or screwing or clamping the remote control 11 onto the nozzle 8.

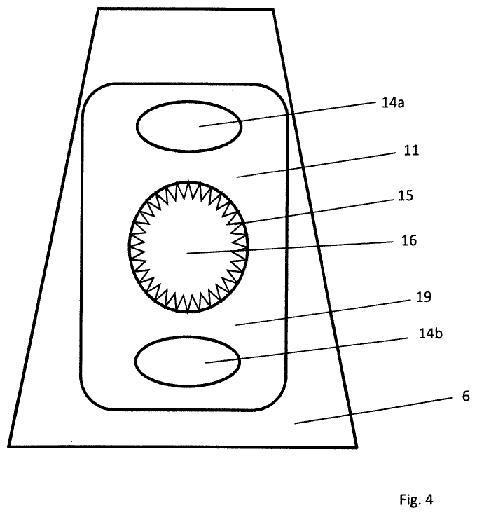

FIG. 4 shows the arrangement of mask or PI and remote control from above. The remote control 11 has a recess 16, which is dimensioned in such a way that the nozzle 8 of the mask can be inserted. For the purposes of affixing the remote control 11 to the nozzle 8, the remote control comprises a plurality of holding elements 15 in the recess 16, said holding elements latching or clamping the remote control 11 onto the nozzle 8. By way of example, the holding elements are molded onto the casing within the scope of an injection molding method from a plastic. Preferably, the holding elements have a lower (Shore) hardness than the casing. The area of the remote control is smaller than the area of the mask. The area of the casing upper side 19 of the remote control is less than 60 cm².

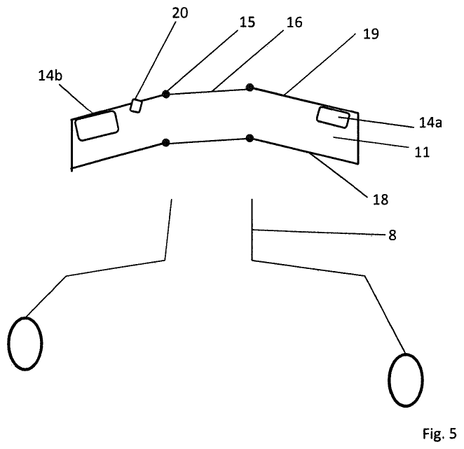

FIG. 5 shows a cross section of the remote control 11. The remote control 11 has an upper side 19 which, for example, is produced integrally in an injection molding method from a hard plastic. Here, in an exemplary manner, the upper side 19 has convex shaping, at least in sections, said convex shaping being adapted to the palm of the hand and therefore being able to be held easily by the hands of the aider. The lower side 18 has shaping which, at least in sections, is adapted to the contour of the mask. The remote control 11 has a lower side 18 which, for example, is produced integrally in an injection molding method from a plastic. Upper side and lower side are preferably produced from a hard plastic. At least one operating element 14 is integrated into the upper side. Either the upper side has recesses for the operating element, into which the operating element is inserted during the assembly, or the upper side is equipped with a manually movable surface in the region of the operating element, said surface being able to be pushed down in order to actuate the operating element lying therebelow. Preferably, the operating element has a surface which is optically and/or haptically distinct from the remainder of the remote control. By way of example, the surface of the operating element 14a, 14b is produced from a soft plastic in the 2K process, said soft plastic being molded onto the hard-plastic casing of the remote control. Alternatively, the operating element can be configured as a touch sensitive (sensor) surface (touchscreen).

Moreover, an indicating means 20 can be provided in the region of the casing of the remote control. The indicating means can be embodied as a display, as a touchscreen or merely as an LED. If the indicating means is a display or a touchscreen, resuscitation parameters are displayable. An LED would visualize functional states of the remote control.

The remote control 11 has a recess/opening 16 in the upper side and/or lower side, said recess/opening being dimensioned in such a way that the nozzle 8 of the mask can be inserted. The remote control 11 has a height which is dimensioned in such a way that the nozzle 8 of the mask passes completely through the remote control and there still is enough free space on the nozzle in order to connect the tube. For the purposes of affixing the remote control 11 to the nozzle 8, the remote control comprises a plurality of holding elements 15 in the recess 16, said holding elements latching or clamping the remote control 11 onto the nozzle 8. The holding elements can be molded onto the casing parts in the 2k process as soft components. Alternatively, the nozzle is conically tapered such that the remote control is clamped onto the nozzle.

The remote control 11 is light and weighs less than 900 grams, preferably less than 700 grams, particularly preferably less than 500 g, very particularly preferably less than 300 grams.

The remote control 11 is small, preferably smaller than the area of the mask, particularly preferably no greater than the area of both hands of the aider. Exemplary values for the area of the remote control are less than 60 cm², preferably less than 50 cm², particularly preferably less than 40 cm².

The height of the remote control 11 is lower than the height of the mask, preferably less than 10 cm, particularly preferably less than 6 cm, very particularly preferably less than 4 cm.

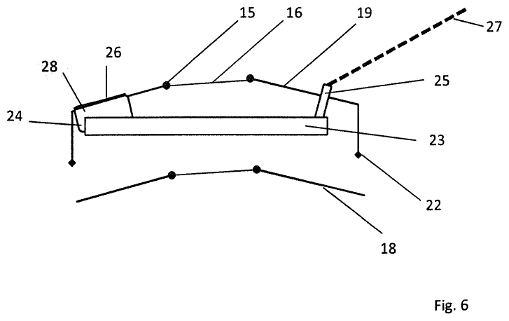

FIG. 6 shows further details of the remote control 11 in a cross section. The casing comprises a casing upper side 19 and a casing lower side 18. In an assembled state, the casing upper side and the casing lower side are connected to one another. In an assembled state, the casing upper side 19 and the casing lower side are connected to one another in a secure and substantially watertight and/or dust-tight manner. The casing upper side has a contact region 22 which is provided for coupling with the casing lower side 18. To this end, the contact region has fastening elements 22, which are provided for interlocking and/or force-fit coupling.

Moreover, the remote control comprises at least one operating element 14. Further, the remote control comprises an electronic printed circuit board or an electronic component 23.

The cable 27 is connected to the component 23. Moreover, the component 23 has electrical contact with the operating element 24.

In an assembled state, the component 23 extends e.g. parallel to a direction of main extent of the casing upper side and it is held in the casing upper side and/or casing lower side in a way that prevents loss. To this end, provision is made of holding elements which are provided for an interlocking and/or force-fit, in particular detachable connection between component and casing upper side.

The operating element 14 has an areal configuration. By way of example, the operating element 14 extends through a wall of the casing upper side 19. In an assembled state, the operating element 14 comprises an interior region 24, an exterior region 28 and an intermediate region. In an assembled state, the exterior region 28 of the operating element 14 can be arranged outside of, or above or level with, the upper side 19 of the casing. In an assembled state, the interior region of the operating element 14 is arranged within the casing 10 and surrounded by the casing.

The exterior region 28 of the operating element 14 has an at least substantially areal configuration, i.e. the exterior region has sections, the directions of the main extent of which are arranged substantially in the direction of the casing upper side. The exterior region 28 of the operating element 14 is fitted and encapsulated or sealed 26 by the casing upper side at at least two edges of a recess. As a result, an interlocking and cohesive affixment of the operating element 14 is obtained relative to the casing upper side 19 of the casing.

The interior region 24 of the operating element comprises a contact region that establishes a connection with a corresponding contact region of the exterior region when the operating element is pushed down. As a result, it is possible to establish an electrical connection between the operating element and the printed circuit board 23.

The sealing unit 26 is provided for a substantially dust-tight and/or watertight connection between the operating element 14 and the casing. The sealing unit has an integral embodiment with the operating element 14 and/or with the casing 19. Alternatively, the sealing unit 26 also has a two-part embodiment with the operating element and/or with the casing, or it is arranged therebetween.

The sealing unit 26 comprises a cohesive connection between the operating element 14 and the casing upper side 19.

The remote control has an electronic printed circuit board or electronic components 23, which, by way of an actuation of the operating element 14, are prompted to transmit control signals to the emergency resuscitation device in order to trigger at least one respiratory gas volume.

In an advantageous configuration, the upper part of the casing 19 consists of a material with a relatively high Shore hardness, into which the operating element has been inserted. A material, e.g. silicone, with a relatively low Shore hardness is pasted or molded over the operating element. While the hard material of the upper part of the remote control provides sufficient dimensional stability, the soft operating element cover facilitates easy actuation.

The remote control can have both a wireless and cable-assisted configuration. A cable 27 guided thereout may be surrounded by a potting material.

If the control signals are transmitted by wire from the remote control to the resuscitation device, the wire connection preferably comprises a data line and an energy line. Control signals are transmitted to the resuscitation device by way of the data line and the remote control is supplied with energy (from the resuscitation device) by way of the energy line.

In an advantageous configuration of the cable-assisted variant, a high-temperature resistant connection cable with a jacket is fastened to the circuit board by means of a cable clamp. Here, the cable clamp brings about strain relief. The connection strands of the cable are soldered onto the printed circuit board. The cable that is guided out of the casing has a kink protector 25.

The control signals of the remote control are transmitted from the remote control to the resuscitation device in a wireless or wired manner. By way of example, the remote control (as a transmitter) is configured as an infrared transmitter or as a radio transmitter or as a radio transmitter of a Bluetooth radio link. The receiver in the resuscitation device has a corresponding configuration and embodiment.

The remote control may also consist merely of an operating film. In accordance with the present invention, the remote control then comprises a passive RFID transponder which, in the case of physical contact by a user, triggers a switching contact in order to emit information items to control the emergency resuscitation device.

Consequently, this can be a very thin flat film button which is adhesively bonded onto a patient interface and which establishes a contact by pressing such that the corresponding chip, which is arranged in the RFID transponder, is activated.

In accordance with a preferred embodiment, the outlet of the cable in the region of the upper side of the casing is considered to be designed in an airtight and/or hydrophobic manner. This can obtain an improved sealing function.

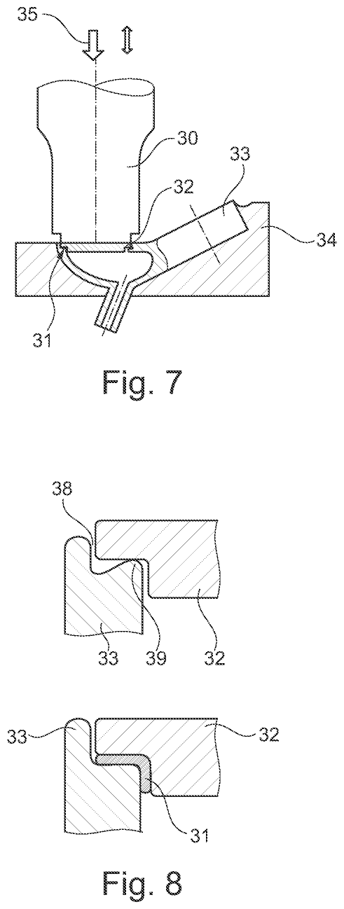

The schematic representation in FIG. 7 shows a device for carrying out a welding process using ultrasound. A sonotrode 30 is positioned in the area of a weld seam 31 to be produced. The weld seam 31 serves to connect a lower housing part 32 of the operating element 4 to an upper housing part 33 of the operating element 4.

The upper housing part 33 is preferably held in a receptacle 34 while the welding process is being carried out. The sonotrode 30 is pressed with a welding force 35.

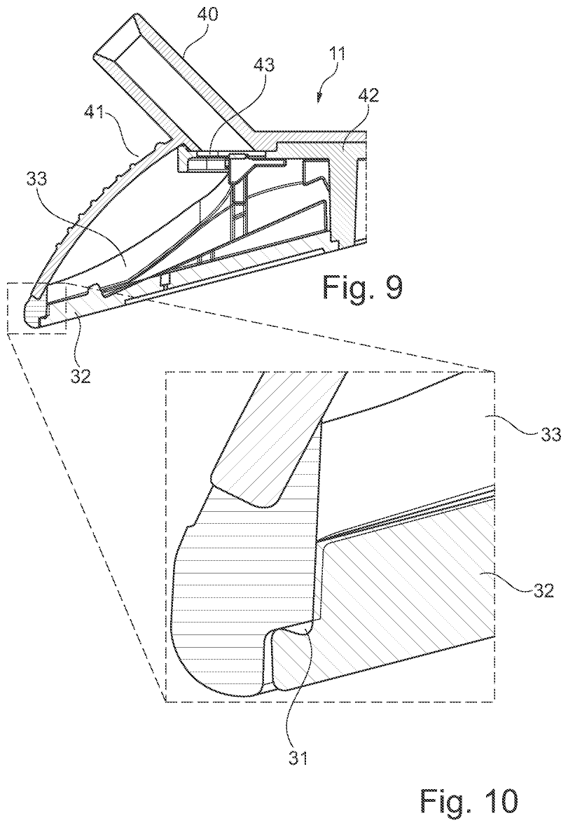

A material projection 43 that can be seen in FIG. 9 consists of the hard material component 42 and, after a cable has been inserted into the cable entry 40, serves to stabilize and hold the cable.

FIG. 10 shows an enlarged partial illustration of FIG. 9 in the area of the weld seam 31. In particular, the material transition from the lower housing part 32 into the weld seam 31 and from there into the upper housing part 33 can be seen.

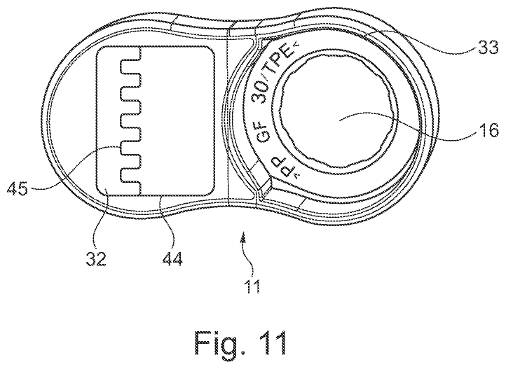

FIG. 11 shows a view of the remote control 11 from below. The upper housing part 33 here surrounds the recess 16 at least in some areas. In the area of the lower part of the housing 32 an approximately rectangular web 44 can be seen, which supports the positioning of a sticker/label, not shown. A groove-like course 45 can also be seen. The course 45 provides a depression which supports ventilation when the lower housing part 32 is combined with the upper housing part 33 and/or when the soft material component 41 is elastically deformed.

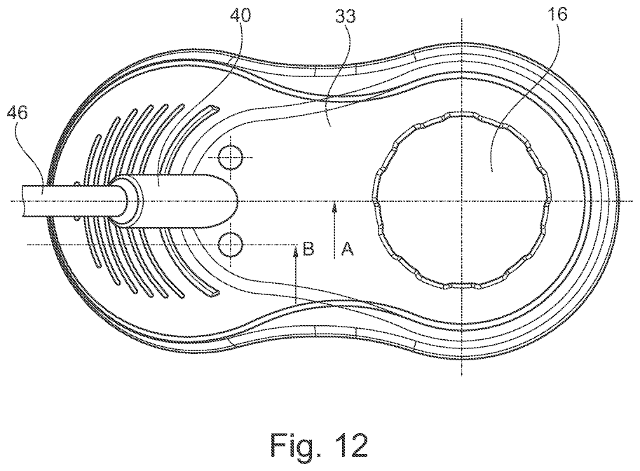

FIG. 12 shows a perspective top view of the upper housing part 33. In particular, it can be seen here that a cable 46 is inserted into the cable entry 40.

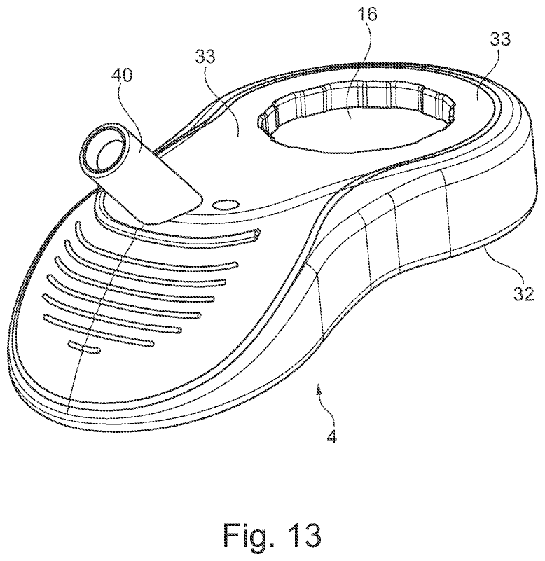

FIG. 13 shows a further perspective illustration of the arrangement according to FIG. 12. The cable 46 is here removed from the cable entry 40 so that a view into the interior of the cable entry 40 is possible.

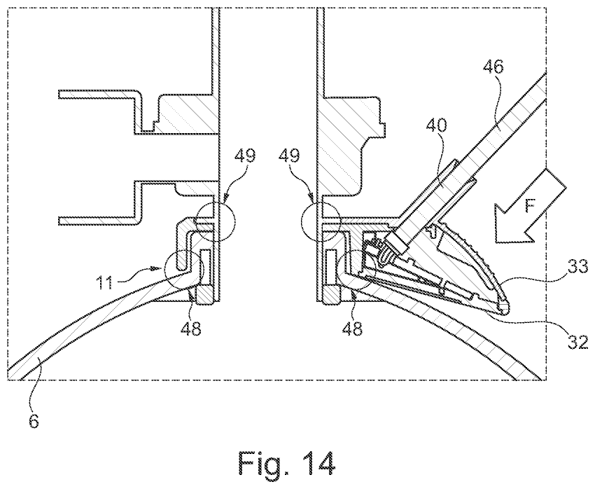

FIG. 14 shows a cross section through a remote control 11 placed on the patient interface 6. A force F is also shown, which often acts on the remote control 11 with an alignment component along the cable 46. With regard to such a force load, the remote control 11 has a collar 48 which is supported on the patient interface 6.

A tight fit of the remote control 11 on the connector 8 is guaranteed even with a corresponding force load. When a corresponding force is exerted, the support provides a rotation area 49.

The welding force 35 is preferably about 280 Newtons. An amplitude when performing the ultrasonic welding is about 92%. A value of approximately 20 kHz has proven to be advantageous as the frequency for the welding. It is also preferred that a time period for carrying out the welding process is a maximum of about 1 second.

A crystalline plastic is preferably used as the material for carrying out the welding process. In particular, the use of polypropylene is intended. A combination of about 70% plastic and 30% glass fibers is particularly preferred as the material for carrying out the welding process. The heat conduction during the welding process can be improved by the glass fibers. The proportions are weight percentages.

Compared to gluing, the welding achieves a higher load capacity of the connection point. In addition, the production time can be shortened and the reproducibility improved. Compared to adhesive bonding, the emission of volatile substances is avoided and the number of different substances used is reduced. This improves the recycling properties.

FIG. 8 illustrates the implementation of a welding of the housing lower part 32 to the housing upper part 33. In the upper part of FIG. 8 the housing parts 32, 33 are shown before the welding process is carried out. A gap 38 can be seen between the housing parts 32, 33, at least in some areas. In the illustrated embodiment, the upper housing part 33 is provided with a material projection 39 in the area of the gap 38. The material projection 39 can be used as a supply of material for carrying out the welding process. In principle, however, it is also possible to return the material separately for performing the ultrasonic welding. It is also possible to provide a corresponding material projection 39 as part of the lower housing part 32.

FIG. 9 shows a vertical section through the remote control 11. In particular, the lower housing part 32 and the upper housing part 33 can be seen. The upper part 33 of the housing is provided with a hollow cylinder-like or sleeve-shaped cable entry 40. In the illustrated embodiment, the upper housing part 33 consists of at least one soft material component 41 and at least one hard material component 42. In this context, soft is understood to mean a consistency similar to rubber-like materials. Under hard a material consistency similar to hard plastics, wood or metal.

The weld seam 31 can be implemented as a closed circuit, for example with a ring-like design. Alternatively, however, separate segments of the weld seam 31 arranged at a distance from one another can also be implemented.

While specific embodiments of the invention have been shown and described in detail to illustrate the inventive principles, it will be understood that the invention may be embodied otherwise without departing from such principles.

We claim:

1. A resuscitation system, comprising: a resuscitation device arranged away from a patient; a patient interface having a connecting tube; a respiratory gas tube that connects the connecting tube to the resuscitation device in an air-conducting manner; and a remote control for the resuscitation device arranged in a region of the patient interface so as to be near the patient, wherein the remote control includes a casing with an upper part and a lower part and the upper part and/or the lower part are made, at least in sections, from hard plastic, wherein the remote control further includes at least one operating element and an electronic printed circuit board or, at least, an electronic component, wherein the electronic component is activated by actuating the operating element in order to produce a control signal for the resuscitation device, wherein the operating element has a surface which, optically and/or haptically, is distinct from a remainder of the remote control, wherein a direction of main extent of the remote control is alignable substantially parallel to a face of the patient, wherein the operating element extends through a wall in the casing upper part, for which purpose the casing has a recess, and the operating element has an interior region and an exterior region, wherein the interior region of the operating element is arranged within the casing and surrounded by the casing, wherein the remote control is detachably connected to the connecting tube of the patient interface, wherein the exterior region of the operating element has a substantially areal configuration and the exterior region of the operating element is fitted into the casing upper part and sealed by a sealing unit so that interlocking and/or cohesive affixment or coverage of the operating element relative to the casing upper part of the casing is achieved and a substantially dust-tight and/or watertight connection is obtained between the operating element and the casing, wherein the interior region of the operating element comprises a contact region that establishes a connection between the operating element and the electronic component when the operating element is pressed down and a control signal is transmitted to the resuscitation device in order to trigger at least one respiratory gas volume, wherein the remote control is connected to the connecting tube by a circular opening in the casing, wherein the circular opening surrounds the connecting tube, further comprising ribs on an inner surface of the circular opening, wherein the casing has a circular collar supported by the patient interface, wherein the upper part and the lower part of the casing are welded together by a plastic material that is liquifiable by ultrasonic waves.

2. The resuscitation system according to claim 1, wherein the upper part and/or the lower part of the casing have a shaping, at least in sections, which is based on a contour of the patient interface or of the hand of a user, wherein the casing upper part has a contact region that is provided for coupling with the casing lower part, wherein the casing upper part and the casing lower part, in an assembled state, are securely connected to one another in a substantially watertight and/or dust-tight manner, wherein the casing of the remote control has a height HF and a length LF, wherein the length LF is greater than the height HF, as a result of which the remote control has a substantially areal configuration and wherein the exterior region of the operating element is situated above, or level with, the upper part of the casing.

3. The resuscitation system according to claim 1, wherein the exterior region of the operating element has a substantially areal configuration and has sections with a main direction of extent arranged substantially in the direction of the casing upper part.

4. The resuscitation system according to claim 1, wherein the sealing unit is integral with the operating element and/or with the casing.

5. The resuscitation system according to claim 1, wherein the sealing unit forms a cohesive connection with the casing upper part and, proceeding from the casing upper part, completely covers the operating element.

6. The resuscitation system according to claim 1, wherein an area of the casing upper part of the remote control is less than 60 cm².

7. The resuscitation system according to claim 1, wherein the upper part of the casing consists of a material with a relatively high Shore hardness, into which the operating element has been inserted, and a material with a relatively low Shore hardness is completely pasted or molded over the operating element.

8. The resuscitation system according to claim 1, wherein the sealing unit is made of a soft plastic using a 2K process, said soft plastic being molded onto the hard-plastic casing of the remote control.

9. The resuscitation system according to claim 1, wherein the operating element is configured as a touch sensitive surface.

10. The resuscitation system according to claim 1, wherein two operating elements are arranged in the casing of the remote control.

11. The resuscitation system according to claim 1, wherein the at least one operating element is configured and embodied in the remote control so that a person affixing the patient interface over airways of the patient with a typical C-grip, is able to actuate the operating element with a lower side of the fingers or a ball of the thumb.

12. The resuscitation system according to claim 1, wherein the remote control has a length that undershoots a length of the patient interface.

13. The resuscitation system according to claim 1, wherein the remote control has a height that is less than a height of the patient interface.

14. The resuscitation system according to claim 1, wherein an assembly consisting of the remote control and the patient interface is only as high as the patient interface on its own.

15. The resuscitation system according to claim 1, wherein the remote control has a recess in the casing, said recess being dimensioned so that the connecting tube of the patient interface can be introduced and the remote control is plugged onto the connecting tube and therefore arranged near the patient interface.

16. The resuscitation system according to claim 1, wherein the remote control has a height which is dimensioned so that the connecting tube of the patient interface passes completely through the remote control and there still is enough free space on the connecting tube in order to connect the tube.

17. The resuscitation system according to claim 15, wherein the remote control comprises a plurality of holding elements in the recess, said holding elements serving to affix the remote control on the connecting tube and latching or clamping the remote control onto the connecting tube.

18. The resuscitation system according to claim 17, wherein the holding elements are molded onto the casing parts as a soft component in the 2k process.

19. The resuscitation system according to claim 1, wherein at least one indicating means is arranged in a region of the casing of the remote control.

20. The resuscitation system according to claim 1, wherein the remote control weighs less than 700 grams.

21. The resuscitation system according to claim 20, wherein the remote control weighs less than 300 grams.

22. The resuscitation system according to claim 1, wherein the remote control transmits the control signal wirelessly to the resuscitation device.

23. The resuscitation system according to claim 1, wherein the remote control transmits the control signal in a wired manner to the resuscitation device and the wire is guided out of the casing in an region of the upper part and the wire is sealed in relation to the casing in a substantially dust-tight and/or watertight manner and the wire has a kink protector adjacent to the casing.

24. The resuscitation system according to claim 25, wherein the wire has an airtight and/or hydrophobic embodiment.

25. The resuscitation system according to claim 1, wherein the lower housing part and the upper housing part are connected to one another by a circumferential weld seam.

26. The resuscitation system according to claim 25, wherein the weld seam is a crystalline plastic.

27. The resuscitation system according to claim 26, wherein the plastic is polypropylene.

28. The resuscitation system according to claim 25, wherein glass fibers are arranged in the weld seam.

29. The resuscitation system according to claim 28, wherein a proportion of glass fibers in the plastic is about 30 percent by weight.

30. A method for connecting a lower housing part of a remote control for a ventilation system with an upper housing part of the remote control, comprising the step of connecting the lower housing part to the upper housing part by ultrasonic welding.

31. The method according to claim 30, wherein the ultrasonic welding has a frequency of approximately 20 kHz.

32. The method according to claim 30, including applying a pressing force of approximately 280 Newtons during the welding.

33. The method according to claim 30, wherein the ultrasonic welding has an amplitude of approximately 92%.

34. The method according to claim 30, wherein the welding has a maximum duration of approximately 1 second.

35. The method according to claim 30, including using a crystalline plastic as material for the ultrasonic welding.

36. The method according to claim 35, including using polypropylene as the plastic.

37. The method according to claim 33, including using a plastic with proportions of glass fibers to carry out the ultrasonic welding.

38. The method according to claim 37, wherein a proportion of glass fibers in the plastic is about 30%.
